(19) United States
(12) Patent Application Publication
(10) Pub. No.: US 2010/0086584 A1
(43) Pub. Date: Apr. 8, 2010

(54) VACCINE COMPOSITIONS OF M2e, HA0 AND BM2 MULTIPLE ANTIGENIC PEPTIDES

(75) Inventors: Bernadette Callejo, Fremont, CA (US); Tom Monath, Harvard, MA (US); Jeffery Fairman, Mountain View, CA (US)

Correspondence Address: DLA PIPER LLP (US), 4365 EXECUTIVE DRIVE, SUITE 1100, SAN DIEGO, CA, 92121-2133, US

(73) Assignee: Juvaris BioTherapeutics, Inc., Burlingame, CA (US)

(21) Appl. No.: 12/563,012

(22) Filed: Sep. 18, 2009

Related U.S. Application Data
(60) Provisional application No. 61/098,005, filed on Sep. 18, 2008.

(51) Int. Cl. A61K 9/127; A61K 39/12; A61K 39/385; A61K 39/145; A61P 31/16

(52) U.S. Cl. 424/450; 424/186.1; 424/196.11; 424/209.1

(57) ABSTRACT

The present disclosure generally relates to a composition comprising one or more peptides selected from influenza virus antigenic peptides M2e, HA0, BM2 and a M2e-BM2 fusion peptide in a composition with a cationic liposome delivery vehicle, and the use of these compositions as a universal vaccine against influenza A and/or B viral strains.

### CROSS REFERENCE TO RELATED APPLICATION

This application claims the benefit of priority under 35 U.S.C. §119(e) of U.S. Ser. No. 61/098,005, filed Sep. 18, 2008 the entire content of which is incorporated herein by reference.

### GRANT INFORMATION

This invention was made in part with government support under Grant No. 1U01AI074512 awarded by the National Institute of Allergy and Infectious Diseases (NIAID). The United States government has certain rights in this invention.

### BACKGROUND OF THE INVENTION

1. Field of the Invention

The invention relates generally to vaccines and more specifically to universal flu vaccines comprising the use of one or more peptides comprising M2e, HA0 and BM2, or one or more fusion peptides created by any combination of amino acids from any of M2e, HA0 and BM2 in a composition with an adjuvant, such as a cationic lipid or liposome delivery vehicle, or cationic lipid DNA complex, and the use of these compositions as a universal vaccine against influenza A and/or B viral strains.

2. Background Information

The conventional vaccine strategy for control of influenza A is vulnerable to antigenic drift and the emergence of unmatched epidemic strains that cause primary vaccine failure. A vaccine strategy that targets an influenza antigen, which is less susceptible to antigenic variation would be a major improvement.

There has been much interest in the development of vaccines that elicit a protective antibody response to the conserved ectodomain of matrix protein 2 (M2e) of influenza A. Another relatively conserved antigenic epitope in influenza A is HA0 which is the cleavage site for hemagglutinin and has been used in preclinical experiments with limited success. BM2 is the homolog of M2e in influenza B virus. Previous development of effective peptide vaccines against these targets has been challenging due to the lack of immunogenicity of the target peptide. The present disclosure covers the use of peptides M2e, HA0, and BM2 individually and in combination in a therapeutic composition comprising a cationic liposome delivery vehicle. The individual and peptide combination compositions may be used to provide a therapeutic effect against influenza A and B viral strains.

### SUMMARY OF THE INVENTION

The present invention includes compositions and methods of using said compositions to provide a therapeutic effect against influenza. More particularly, the present invention relates to methods and compositions for a universal flu vaccine. The present disclosure provides for the use of one or more peptides comprising M2e, HA0 and BM2 in composition with an adjuvant, such as a cationic liposome delivery vehicle or a cationic lipid DNA complex, to vaccinate a mammalian subject against the effects of influenza A or B viral strains.

Embodiments of the present invention feature a composition useful for vaccinating a mammalian subject against influenza virus comprising one or more multiple antigenic peptide sequences formulated with a cationic liposome delivery vehicle.

Compositions contemplated for vaccinating a mammalian subject against influenza A, influenza B or both influenza A and B may feature multiple antigenic peptide M2e conjugated with a cationic liposome delivery vehicle. The compositions may further comprise multiple antigenic peptides HA0 and BM2 or may feature a fusion peptide comprising amino acids from M2e and BM2.

Another composition contemplated for vaccinating a mammalian subject against influenza A influenza B or both influenza A and B includes multiple antigenic peptide HA0 conjugated with a cationic liposome delivery vehicle. The composition may further comprise multiple antigenic peptides M2e and BM2 or may feature a fusion peptide comprising amino acids from more than one antigenic peptide.

Compositions contemplated for vaccinating a mammalian subject against influenza B, influenza A, or both influenza A and B may feature multiple antigenic peptide BM2 conjugated with a cationic liposome delivery vehicle. The compositions may further comprise multiple antigenic peptides HA0 and M2e or may feature a fusion peptide comprising amino acids from M2e and BM2.

Additional embodiments of the featured compositions may include liposome delivery vehicles comprising lipids selected from the group consisting of multilamellar vesicle lipids and extruded lipids.

Additional liposome delivery vehicle embodiments may include pairs of lipids selected from the group consisting of DOTMA and cholesterol; DOTAP and cholesterol; DOTIM and cholesterol; and DDAB and cholesterol.

Additional embodiments feature methods of vaccinating a mammalian subject against influenza virus by administering one of the compositions embodied in the present invention.

### BRIEF DESCRIPTION OF THE DRAWINGS

The accompanying drawings, which are incorporated in and form a part of the specification, illustrates embodiments of the present invention, and together with the description serve to explain the principles of the invention.

In the Drawings:

FIG. 1 is an illustration of the influenza A virus and shows the interaction of Multiple Antigenic Peptides (MAP) within.

FIG. 2 shows survival following M2e-MAP-4/JVRS-100 vaccination and lethal influenza A challenge.

FIG. 3 shows body weight loss following PR/8/34 lethal challenge of vaccinated mice.

FIG. 4 shows the M2e-specific Total IgG sera titer in mice.

FIG. 5 shows the M2e-specific IgG1 and IgG2a Sera titer in mice.

FIG. 6 shows the Lung Lesion analysis for M2e administered with and without a liposome delivery vehicle (JVRS-100).

FIG. 7 illustrates the action steps for the Adoptive Transfer Technique.

FIG. 8 shows Serum Transfer Protection with JVRS-100/M2e with percent survival and body weight.

FIG. 9 shows survival following HA0-MAP/JVRS-100 Vaccination and PR/8/34 lethal Influenza A challenge.

FIG. 10 shows body weight loss following HA0-MAP/JVRS-100 Vaccination and PR/8/34 lethal challenge of vaccinated mice.

FIG. 11 shows M2e-MAP-4 specific IgG in mice after receiving serum transfer and lethal challenge with PR/8/34.

FIG. 12 shows survival (left side) and body weight (right side) following M2e-MAP4/JVRS100 Vaccination and lethal H1N1 (PR/8/34) challenge.

FIG. 13 shows survival (left side) and body weight (right side) following M2e-MAP4/JVRS100 Vaccination and lethal H3N2 (HK×31) challenge.

FIG. 14 photographically shows the Lung Pathology found in the lung tissue following M2e-MAP4/JVRS100 Vaccination and lethal H3N2 (HK×31) challenge.

FIG. 15 shows body weight associated with a range of doses of M2e-MAP4/JVRS100 Vaccination and lethal H1N1 (PR/8/34) challenge.

FIG. 16 shows survival associated with a range of doses of M2e-MAP4/JVRS100 Vaccination and lethal H1N1 (PR/8/34) challenge.

FIG. 17 shows body weight associated with a range of doses of M2e-MAP4/JVRS100 Vaccination and lethal H1N1 (PR/8/34) challenge.

FIG. 18 shows results of a competitive binding ELISA comparing M2e/JVRS with M2e-MAP-4, and M2e-MAP4/JVRS.

FIG. 19 shows the antibody response in mice vaccinated once with M2e-MAP4/TIV/JVRS-100.

FIG. 20 shows body weight following M2e-MAP4, Fluzone, Fluzone/JVRS 100, and Fluzone/M2e-MAP-4/JVRS-100 Vaccination and lethal H3N2 (HK×31) challenge.

FIG. 21 shows survival following M2e-MAP4, Fluzone, Fluzone/JVRS 100, and Fluzone/M2e-MAP-4/JVRS-100 Vaccination and lethal H3N2 (HK×31) challenge.

FIG. 22 shows survival following M2e-BM2 fusion peptide vaccination with and without JVRS-100 and lethal H3N2 (HK×31) challenge.

FIG. 23 shows body weight following M2e-BM2 fusion peptide vaccination with and without JVRS-100 and lethal H3N2 (HK×31) challenge.

FIG. 24 shows survival following M2e-BM2 fusion peptide vaccination with and without JVRS-100 and lethal H1N1 (PR/8/34) challenge.

FIG. 25 shows body weight following M2e-BM2 fusion peptide vaccination with and without JVRS-100 and lethal H1N1 (PR/8/34) challenge.

FIG. 26 shows survival following M2e-BM2 fusion peptide vaccination with and without JVRS-100 and lethal 200 HA B/Malaysia challenge.

FIG. 27 shows body weight following M2e-BM2 fusion peptide vaccination with and without JVRS-100 and lethal 200 HA B/Malaysia challenge.

### DETAILED DESCRIPTION OF THE INVENTION

The present disclosure generally relates to a composition comprising one or more peptides selected from M2e, HA0 and BM2 or fusion peptides of any combination of the M2e, HA0 or BM2 peptides, in a composition with a cationic liposome delivery vehicle, and the use of these compositions as a universal vaccine against influenza A and/or B viral strains.

Use of a conventional vaccine strategy for control of influenza A may lead to primary vaccine failure because of vulnerability to antigenic drift and emergence of unmatched epidemic strains. A vaccine strategy employing an influenza antigen which is less susceptible to antigenic variation would be a major improvement. Several vaccines have been developed and tested clinically and pre-clinically using the M2e peptide as a basis for broad-based protection. The native M2e is a 23-amino acid long ectodomain of the Matrix protein 2 (M2) which is vastly conserved amongst human influenza A virus strains. In contrast to other approaches which have presented the M2e as a monomer or string of monomers, the present invention utilizes a synthetic M2e-peptide constructed in a multiple antigenic peptide which may be MAP-2 or MAP-4, with MAP-4 most preferred. This orientation of the antigen assumes a tetrameric form much like the native form of the M2 protein in the virus or infected cells. When used with a cationic lipid DNA complex adjuvant, such as (JVRS-100 adjuvant), the M2e-MAP4 is presented to the immune system it in a much more immunogenic form. Vaccination with M2e-MAP4/JVRS-100 resulted in a significant increase in total IgG, IgG1 and IgG2a M2e-specific antibodies compared with unadjuvanted M2e-MAP4 alone. As shown in previous studies with other antigens, JVRS-100 increased the T_{H}1 bias indicated by production of significant amounts of anti-M2e IgG2a compared with IgG1. This exemplifies that the mechanism of protection of M2e vaccinated mice may be NK mediated antibody-dependent cellular cytotoxicity (ADCC) and IgG2a antibodies bind tightly to the FcγRIII of NK cells. The addition of JVRS-100 adjuvant protected mice from lethal challenge against H1N1 and H3N2 strains in terms of survival and improved morbidity. The adjuvanted M2e-based vaccine provides protective immunity primarily due to a humoral response which is transferable by serum. There is 100% protection from mortality at peptide vaccine doses of M2e-MAP4 of 100, 50, and 25 ng. Additionally, M2e-MAP4/JVRS-100 vaccine may be used as an additive to traditional seasonal influenza vaccine to protect against drifted strains.

Use of a partially purified split vaccine for control of influenza A has repeatedly caused primary vaccine failures due to the emergence of antigenic variants that are poorly matched to virus strains in the vaccine. The recent occurrence of a pandemic caused by novel H1N1 (swine origin) is a dramatic case in point. A vaccine strategy employing an influenza antigen which is less susceptible to antigenic variation would be a major improvement. Although other proteins of fluA, such as the nucleoprotein have been investigated as “universal” antigens, M2e remains the most effective vaccine candidate. The approach of the present invention includes a cationic lipid-DNA complex adjuvant (A/RS-100) with the M2e-MAP4 without the use of T-cell helper peptides. This complex effectively delivers the antigen to APCs and presents the antigen in a much more immunogenic form. The antigen contribution to the improved response may be a consequence of the orientation of the antigen in the native M2e tetrameric form, while the adjuvant contribution may also play a role in the antigen orientation and results in a predominance of IgG2 (T_{H}1 biased antibody) which has been demonstrated to be more effective via ADCC than IgG1 (T_{H}2-biased antibody).

Embodiments of the present invention feature an adjuvanted M2e vaccine based on a multiple antigen peptide configuration with a strong T_{H}1 adjuvant that can be used either alone or in combination with seasonal influenza vaccination.

Influenza A

Influenza A is an enveloped negative single-stranded RNA virus that infects a wide range of avian and mammalian species. Human infection mainly involves the upper and lower respiratory epithelial tracts, with approximately 20% of children and 5% of adults worldwide experiencing symptomatic influenza each year. During an average epidemic season in the United States, an influenza season of typical severity results in >100,000 cases requiring hospitalization and >30,000 deaths, with 90% of the morbidity and mortality occurring in the elderly (≧65 years of age).

Influenza A is classified into serologically defined antigenic subtypes of the hemagglutinin (HA) and neuraminidase (NA) surface glycoproteins. Sixteen HA and 9 NA influenza A subtypes have been serologically identified. Most Influenza A subtypes are carried asymptomatically in the gastrointestinal tract of wild birds but some may cause disease in domestic birds or mammals. Since the beginning of the twentieth century, only H1N1, H3N2, and H2N2 have caused recurrent human annual epidemics.

The genome consists of eight negative-sense ssRNA molecules. HA mediates viral attachment to terminal sialic acid residues on host cell glycoproteins and glycolipids. HA is involved in viral fusion with the cell membrane and NA cleaves terminal sialic acid residues of influenza A cellular receptors required for the release and spread of mature virions and is the target of inhibitor drugs-such as oseltamivir phosphate (Tamiflu™). A single RNA segment encodes two matrix proteins, M1 and M2. M1 is located immediately below the lipid bilayer of the virus, and M2 serves as an ion channel that has a small extracellular surface domain. Another RNA segment encodes NS-1, which counteracts the host cell type I interferon (IFN) production, and nuclear export protein, which facilitates RNA nuclear export. The other four segments encode the PB 1, PB2, and PA polymerases for viral transcription and nucleoprotein (NP), which encapsulates the genomic RNA segments.

Antigenic Shift and Drift

Segmentation of the influenza A genome facilitates its reassortment when two or more strains infect the same cell yielding major genetic changes, called antigenic shifts. Antigenic shifts caused two major influenza A pandemics of the twentieth century, the 1957H2N2 (Asian flu) and 1968H3N2 (Hong Kong flu) outbreaks. A third mild pandemic, which was due to the reappearance of a H1N1 substrain in 1977 that was absent from circulation since 1950, was most likely reintroduction of a previously frozen laboratory strain as part of a military vaccination experiment. Antigenic drift is the accumulation of viral strains with minor genetic changes, mainly amino acid substitutions. The virus-encoded RNA-dependent RNA polymerase complex is relatively error-prone (˜1/104 bases per replication cycle) and these point mutations are the major source of antigenic drift. Selection favors the circulation of influenza A strains with antigenic drift and shift involving the HA and NA because this allows strains to avoid the impact of neutralizing antibodies that inhibit viral attachment to host cells. Antigenic drift accounts for the annual nature of flu epidemics, and also explains the reduced efficacy of vaccination, which is based on neutralizing antibody if the amino acid sequence of the HA protein used in vaccination does not match that encountered during the epidemic.

M2e Vaccine

Natural infection with influenza does not induce a robust immune response against M2e. This fact has stimulated considerable interest in artificial immunization against M2e as a means of evoking cross-protective immunity in humans. The M2-protein is a tetrameric transmembrane protein present on influenza A viral particles and on virus-infected cells. The ectodomain of the M2-protein is 23 amino acids in length and has remained reasonably unchanged since the isolation of the first influenza strains in 1933. Therefore, there has been significant interest in development of an M2e based universal influenza vaccine.

The main impediment for development of an M2e peptide based vaccine has been the production of a robust immune response to the M2e epitope following vaccination. To increase immunogenicity of the M2e peptide various groups have evaluated adjuvants and antigen presentation techniques. Previous investigators have demonstrated that the M2e sequence conjugated to or genetically fused to carrier proteins providing T cell help, including Hepatitis B core (HBc) protein, Salmonella flagellin, or the outer membrane protein of Neisseria meningitides increased the immunogenicity of the M2e epitope. While these studies showed efficacy in murine studies, the M2e protein was presented as a monomer or as a tandem repeat structure rather than in the tetrameric form of the native M2e thereby limiting the recognition of conformational epitopes formed by multiple copies of the M2e peptide. DeFilette and colleagues have subsequently investigated a modified form of the leucine zipper of the yeast transcription factor GCN4 linked to M2e. This chimeric protein mimics the quaternary structure of the ectodomain of the natural M2 protein and has shown recognition of conformational epitopes which may be critical for enhanced protection with M2e. M2e epitope coupled with Neisseria meningitidis outer membrane complex (OMPC) has shown considerable immunogenicity in preclinical models, although it is unclear if such a chimeric protein approach will be feasible for repeated yearly vaccination given the immunogenicity of the carrier protein. Approaches with chimeric proteins have significant disadvantages by the elicitation of antibody and in some cases T-cell responses which are non-protective versus influenza A and may result in a decreased response to repeated vaccination which is essential for annual seasonal influenza vaccination.

The use of multiple antigenic peptides (MAPs) where copies of M2e are synthesized with helper T cell peptides has also been investigated. While these studies showed promise in early murine studies, only 15% of the M2e-specific Abs cross-reacted with M2e expressed by M2-transfected cells suggesting a lack of recognition of conformational epitopes. This lack of affinity for cells with transfected M2 was also observed when the M2e-MAP was used with immunostimulatory oligodeoxynucleotide 1826 (ODN) or ODN and cholera toxin (CT) adjuvant.

In contrast to other approaches for producing an efficacious M2e based vaccine present embodiments of the disclosure utilize a M2e-MAP4 configuration which has no targeting moieties and thus is more likely to attain a tetrameric conformation similar to native M2e. This M2e-MAP4 is combined with a cationic lipid DNA complex adjuvant such as JVRS-100 which further facilitates effective antigen presentation similar to the native M2e in the membrane of infected cells and specifically targets the M2e-MAP4 to dendritic cells for antigen presentation. The examples representing embodiments of the present invention demonstrate an enhanced immune response and protection from infection that when using the antigen/adjuvant combinations contemplated in the present invention.

Adjuvants

Cationic liposome/DNA complexes (CLDCs such as JVRS-100) were originally developed as a gene delivery system for the delivery of bacterial plasmid DNA for potential gene therapy. The administration of JVRS-100 activated innate immunity and inhibited gene expression. JVRS-100 administration resulted in the release of particularly high circulating levels of IFN-α, suggesting potent activation of pDCs, and IL-12, suggestive of cDC activation. This activation was independent of whether the plasmid contained any cDNA coding region (the ‘empty-vector’ effect) and has subsequently been shown to occur with TLR3 agonists as well when the same mixture of cationic and neutral lipids are used. The addition of peptide or protein antigens to JVRS-100 creates a very potent adjuvant effect with elicitation of strong T-cell and antibody responses. Embodiments of the present disclosure include a TVRS-100-adjuvanted M2e vaccine which may be used alone or as an additive to seasonal flu vaccine which would exploit the T_{H}1 bias of the humoral immune response to induce more efficient and broadly protective vaccinate. The robust antibody response would be advantageous in situations of a vaccine mismatch or emergence of endemic or pandemic influenza.

An embodiment of the present disclosure comprises a composition useful for vaccinating a mammalian subject against influenza virus comprising one or more multiple antigenic influenza virus peptide sequences formulated with a cationic liposome delivery vehicle.

An embodiment of the present disclosure comprises a composition useful for vaccinating a mammalian subject against influenza A comprising multiple antigenic peptide M2e conjugated with a cationic liposome delivery vehicle. The cationic liposome delivery vehicle may be NRS-100. Additional embodiments could include the addition of MA0 or BM2, or both MA0 and BM2. An additional embodiment may include SEQ ID No:1 as the M2e peptide.

An embodiment of the present disclosure comprises a composition useful for vaccinating a mammalian subject against influenza A comprising multiple antigenic peptide HA0 conjugated with an cationic liposome delivery vehicle. Additionally, the HA0 peptide sequence may comprise SEQ ID NO: 2 or 3. The composition may additionally include M2e, or BM2 or M2e and BM2.

An embodiment of the present disclosure comprises a composition useful for vaccinating a mammalian subject against influenza B comprising BM2 conjugated with a cationic liposome delivery vehicle. Additionally, the BM2 protein sequence may comprise SEQ ID NO: 4. The compositions may additionally include M2e, or HA0, or M2e and HA0.

An embodiment of the present disclosure comprises a composition useful for vaccinating a mammalian subject against influenza A and/or B comprising a fusion peptide comprising 10-22 amino acids native to M2e with 5-10 amino acids native to BM2. A preferred fusion peptide comprises 16 amino acids from M2e and 7 amino acids from BM2 and is represented by SEQ ID NO:5. The fusion peptides are conjugated with a cationic liposome delivery vehicle.

An embodiment of the present disclosure comprises a method for vaccinating a mammalian subject against influenza virus comprising administering one or more multiple antigenic influenza virus peptide sequences formulated with a cationic liposome delivery vehicle.

An embodiment of the present disclosure comprises a method for vaccinating a mammalian subject against influenza A virus comprising administering a vaccine composition comprising multiple antigenic peptide M2e conjugated with a cationic liposome delivery vehicle. The cationic liposome delivery vehicle may be JVRS-100. Additional embodiments could include the addition of MA0 or BM2, or both MA0 and BM2. An additional embodiment may include SEQ ID NO:1 as the M2e peptide.

An embodiment of the present disclosure comprises a method for vaccinating a mammalian subject against influenza A virus comprising administering multiple antigenic peptide HA0 conjugated with an cationic liposome delivery vehicle. Additionally, the HA0 peptide sequence may comprise SEQ ID NO: 2 or 3. The composition may additionally include M2e, or BM2 or M2e and BM2.

An embodiment of the present disclosure comprises a method for vaccinating a mammalian subject against influenza B virus comprising administering BM2 conjugated with a cationic liposome delivery vehicle. Additionally, the BM2 protein sequence may comprises SEQ ID NO: 4. The compositions may additionally include M2e, or HA0, or M2e and HA0.

An embodiment of the present disclosure comprises a method for vaccinating a mammalian subject against influenza A and/or B comprising administering a fusion peptide conjugated with a cationic liposome delivery vehicle. Further embodiments utilize a fusion peptide comprising 10-22 amino acids native to M2e with 5-10 amino acids native to BM2. A preferred fusion peptide comprises 16 amino acids from M2e and 7 amino acids from BM2 and is represented by SEQ ID NO:5.

An embodiment of the present disclosure comprises a method for vaccinating a mammalian subject against influenza virus comprising administering one or more multiple antigenic peptide sequences formulated with a cationic liposome delivery vehicle.

An embodiment of the present disclosure comprises a method for vaccinating a subject against influenza A or influenza B with a composition comprising one or more peptides selected from M2e, HA0 and BM2 formulated with a cationic liposome delivery vehicle.

The examples herein are meant to exemplify the various aspects of carrying out the invention and are not intended to limit the invention in any way.

M2e

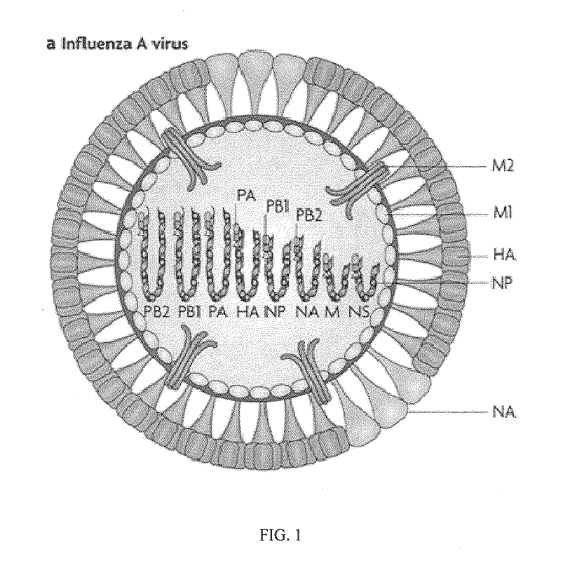

As shown in FIG. 1 M2e is found in an external domain of the M2 protein of Influenza A. It is highly conserved in all known human influenza strains. The MAP-4 peptide is a synthetic peptide containing four copies of M2e.

M2e Experiments

Balb/c mice were vaccinated three times with the M2e peptide in the context of a multiple antigenic peptide (MAP) complex and combination with the cationic lipid DNA complex adjuvant (JVRS-100). Antibody titers were monitored over the time course of vaccination and 2-4 weeks following the final vaccination the mice received a lethal challenge H1N1 (PR/8/34). Vaccination of mice were JVRS-100-MAP4-M2e compared to MAP4-M2e resulted in increased survival, decreased weight loss, and higher recovery following lethal challenge with H1N1 (PR/8/34). Furthermore, recipients of adoptive transfer of serum from MAP-4-M2e/JVRS-100 vaccinated mice demonstrated protection against lethal challenge and weight loss compared to control mice. These studies demonstrate that a simple, fully synthetic vaccine in a multiple antigenic peptide configuration with a strong adjuvant may result in a viable vaccine candidate for universal influenza vaccination and other peptide based vaccine approaches.

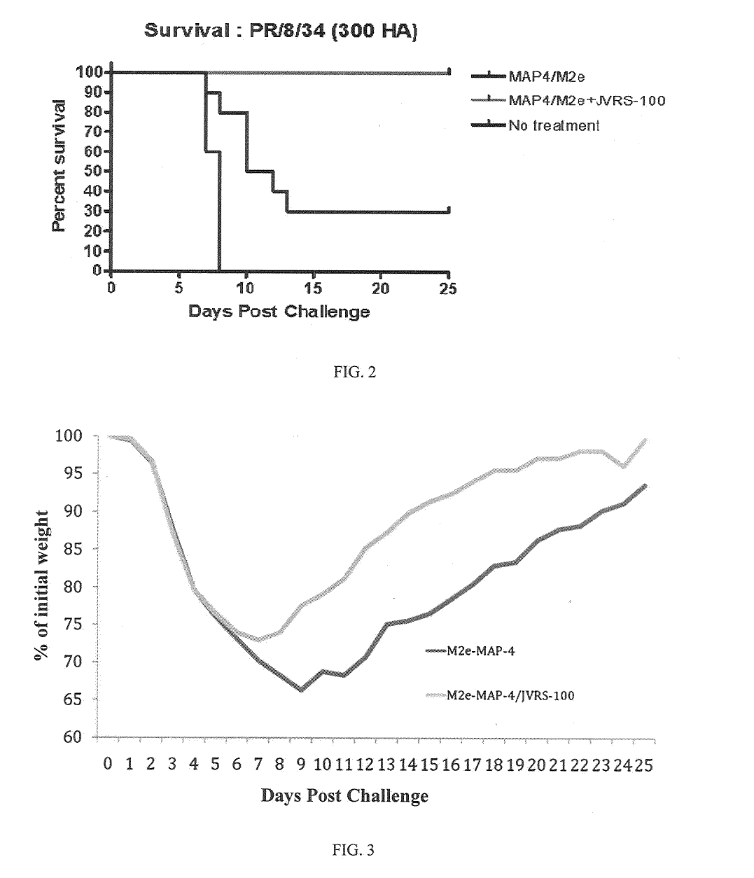

Groups of 10 mice were vaccinated at two week intervals with 54 MAP-4 M2e or 5 ug MAP-4 M2e with cationic lipid DNA Complex (CLDC) adjuvant, sometimes referred to as JVRS-100. At two weeks following the last immunization mice were challenged with 6×LD50 of PR/8/34 and monitored for survival (See FIG. 2) and weight loss (See FIG. 3). Adjuvanted MAP-4/M2e vaccinated conferred 100% protection as compared with unadjuvanted vaccine at 30%. The result was statistically significant (P=0.002). FIG. 2 illustrates survival following M2e-MAP-4/JVRS-100 Vaccination and lethal Influenza A challenge. As shown in FIG. 3, adjuvanted MAP-4/M2e vaccinated mice began to gain weight at 7 days post infection whereas unadjuvanted MAP-4/M2e mice began to gain weight at 9 days post-infection (3 of 10 survivors). At the conclusion of the experiment the adjuvanted group had regained 100% of pre-challenge body weight, whereas the nonadjuvanted group remained at 93%. In the mouse model of influenza challenge, body weight is the accepted clinical sign of morbidity (sickness).

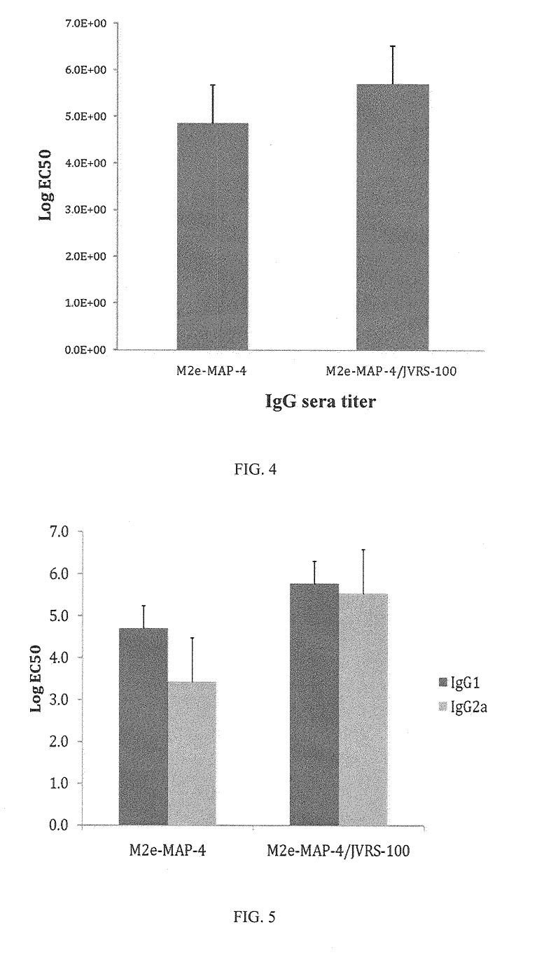

Additionally M2e-specific Total IgG sera titer was determined from the animals in the above study and graphically demonstrated in FIG. 4. The specific anti-M2e IgG level following vaccination was greatest with the MAP-4/M2e+JVRS-100 as compared with MAP-4/M2e alone or with published values (Vaxxinate M2e coupled flagellin). The higher the antibody titer, presumably the more robust the protection. This was assessed 2 weeks following the last of three vaccinations.

FIG. 5 illustrates the M2e-specific IgG1 and IgG2a Sera titer, wherein anti-M2e IgG1 and IgG2a were increased in adjuvanted vaccination compared with unadjuvanted. This was assessed 2 weeks following the last of three vaccinations. Furthermore the addition of JVRS-100 to a candidate vaccine has been shown to increase the TH1 bias of the antibody response. While it has not been shown by direct evidence in these experiments, it is plausible that IgG2a provides greater protection in vivo due to its superior ability to bind to Fc receptors which may play a role in defense against influenza (Huber et al., J Immunol. 166: 7381-7388, 2001). Moreover, IgG2a also is more effective at activating complement than IgG1, and such activation may enhance viral neutralization (Beebee et al., J Immunol. 130: 1317-1322, 1983).

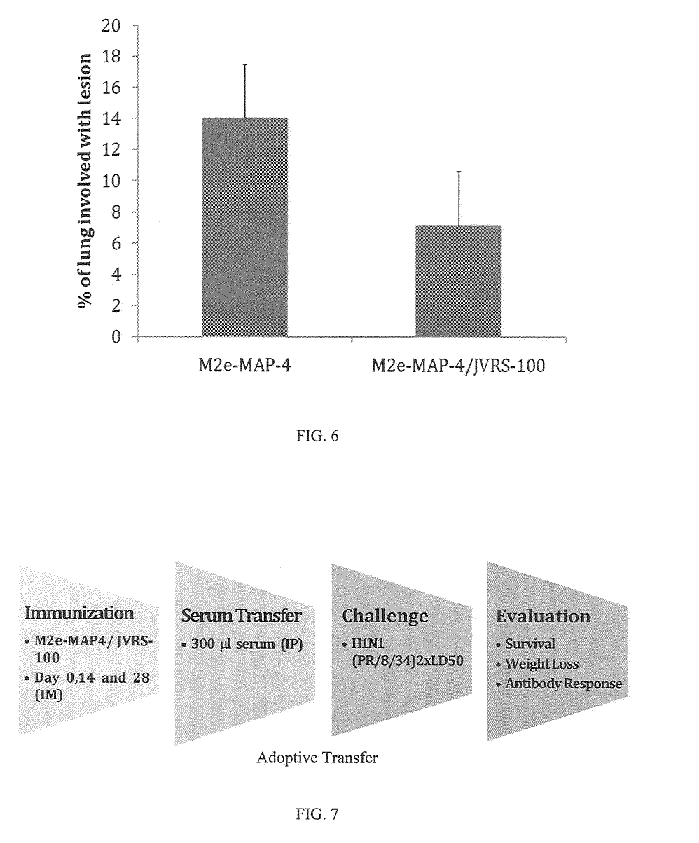

The lung lesion analysis from the above experiment (shown in FIG. 6) represents an evaluation of lung lesions of the mice 25 days post challenge. Three mice from each group were evaluated and the JVRS-100 mice showed considerably lower lung lesions than the unadjuvanted group.

M2e Adoptive Transfer

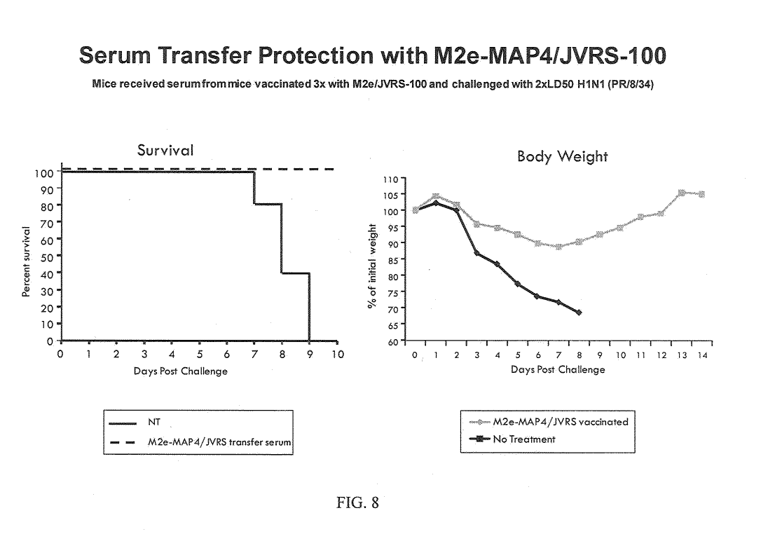

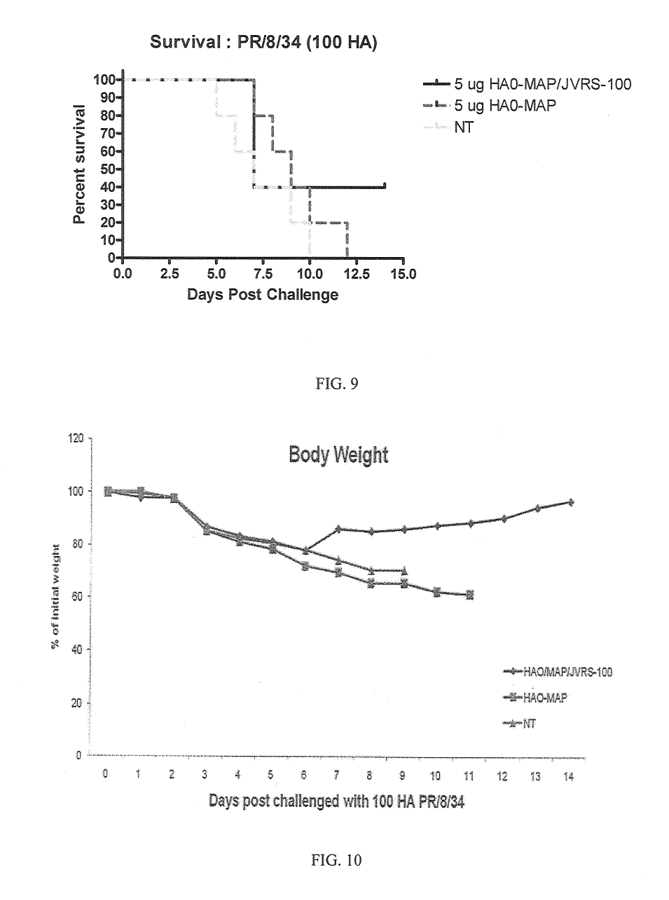

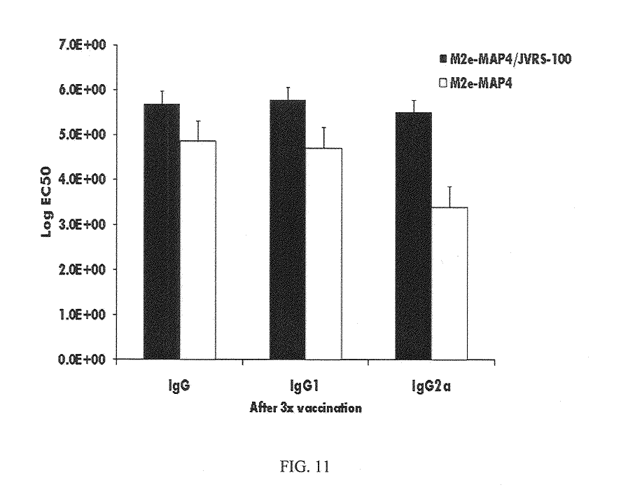

The steps of adoptive transfer are outlined in FIG. 7. The results of the Adoptive transfer experiments are demonstrated in FIGS. 8 and 9. FIG. 8 shows that the recipients of 300 μl serum from MAP-4-M2e/JVRS-100 vaccinated mice and challenged with H1N1 (2×LD50 PR/8/34) resulted in survival of 100% (P=0.0026) compared with control mice which received 200 μl naïve serum (0% survival). This shows that the protection via M2e was primarily antibody mediated. Serum was administered IP and mice were challenged one day later.

FIG. 9 demonstrates M2e-MAP-4 specific IgG in mice after receiving serum transfer and lethal challenge with PR/8/34. The results show that prior to challenge mice had similar IgG2a levels. Following challenge the mice had lower IgG2a levels. This suggests that IgG2a has a higher affinity for virus and is more effective in promotion of ADCC to kill infected cells.

HA0 Experiments

Groups of 5 mice were vaccinated at Day 0, 14 and 28 with 5 ug HA0-MAP/JVRS-100, 5 ug HA0-MAP, or left untreated. At two weeks following the last immunization mice were challenged with 100 HA PR/8/34 and monitored for survival (See FIG. 9) and weight loss (See FIG. 10).

M2e Specific Antibody Response Following Vaccination with M2e-MAP4/JVRS-100

One of the major obstacles to the development of an M2e-based vaccine is that the peptide itself is relatively non-immunogenic. Prior to in-vivo challenge studies the immunogenicity of the M2e-MAP4 and M2e-MAP4/JVRS-100 vaccines was assessed. Mice were vaccinated at day 0, 14, and 28 with 5 μg M2e-MAP4 alone or with 20 μg of JVRS-100. Serum was collected at day 42 and analyzed for IgG, IgG1 and IgG2a antibody titer. Shown in FIGS. 4 and 5 is the relative titer expressed as Log(EC50) of the titration curve. The addition of the JVRS-100 adjuvant resulted in an approximately 10-fold increase in IgG (shown in FIG. 4) and the IgG1 (see FIG. 5) and 100 fold increase in IgG2a (See FIG. 5). Moreover, the antibody response to M2e-MAP4/NRS-100 was >100-fold greater than adjuvanted native M2e peptide (data not shown) indicating the contribution of the adjuvant and antigen to the increase in immunogenicity of the candidate vaccine.

Comparison of immune responses from the literature is always a challenge for the vaccine field given the various methods of assay and calculation of antibody titer. We chose to use the midpoint of the dilution curve since we believe that this measurement is less perturbed by matrix effects such as other proteins and variability in salt concentrations (among other variables). When comparing with published endpoint titers in response to other M2e based vaccines, these responses can be estimated to be 10× greater than other published data. In addition it is important to note that in these previous studies that the IgG2/IgG1 ratio was approximately 0.1 compared to 0.7 shown in FIGS. 4 and 5 indicating the potential for enhanced activity via NK-mediated ADCC.

Protection of M2e-MAP4/JVRS-100 Vaccinated Mice from Lethal Influenza Virus Challenge

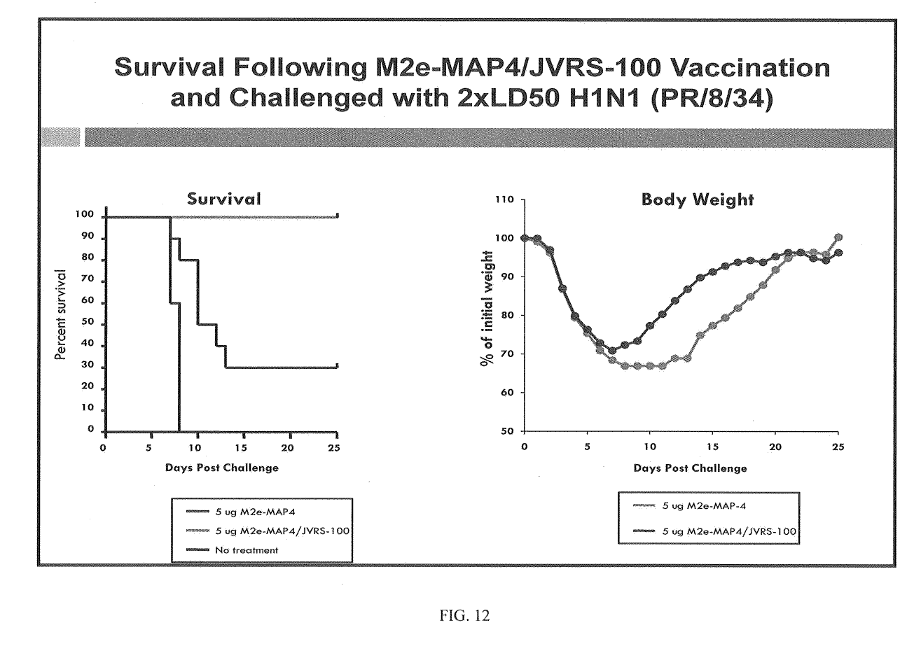

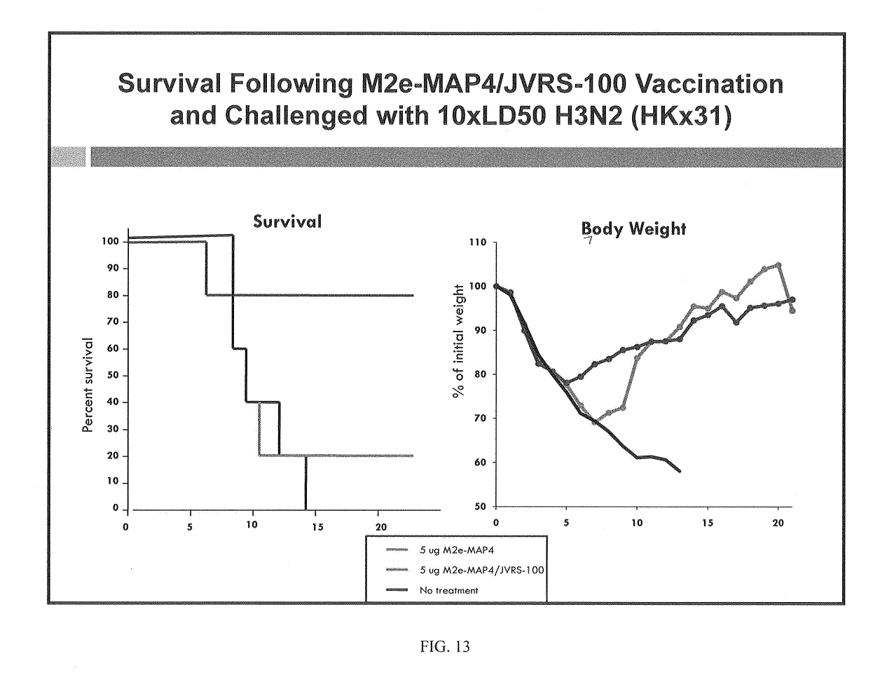

To show the efficacy of the M2e-MAP4/JVRS-100 vaccine, mice were vaccinated on day 0, 14, and 28 and challenged with lethal doses of either a mouse-adapted H1N1 (PR/8/34) or H3N2 (HK×31). While the M2 protein for both isolates was derived from the parent PR/8/34, the isolates had different hemagglutinin and neuraminidase and demonstrated differences in disease course and lethality following serial passages in mice. Therefore, the protection from both viral isolates was evaluated to ensure that there was no change in the efficacy of M2e-MAP4/JVRS-100 vaccination. In these studies mice were vaccinated with M2e-MAP4 with or without JVRS-100 on day 0, 14, 28, and subsequently challenged intranasally with either 2×LD₅₀ of H1N1 (PR/8/34) (shown in FIG. 12) or 10×LD₅₀ of H3N2 (HK×31) (Shown in FIG. 13) viral isolates. As can be seen in FIGS. 12 and 13, there is a significant decrease in morbidity and mortality in the mice vaccinated with M2e-MAP4/JVRS-100.

Lung Pathology of M2e-MAP4/JVRS-100 Vaccinated Mice Following Lethal Influenza Virus Challenge

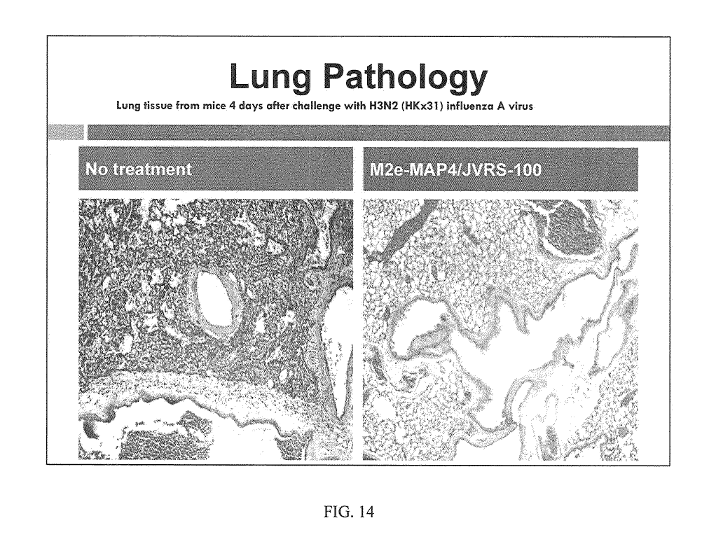

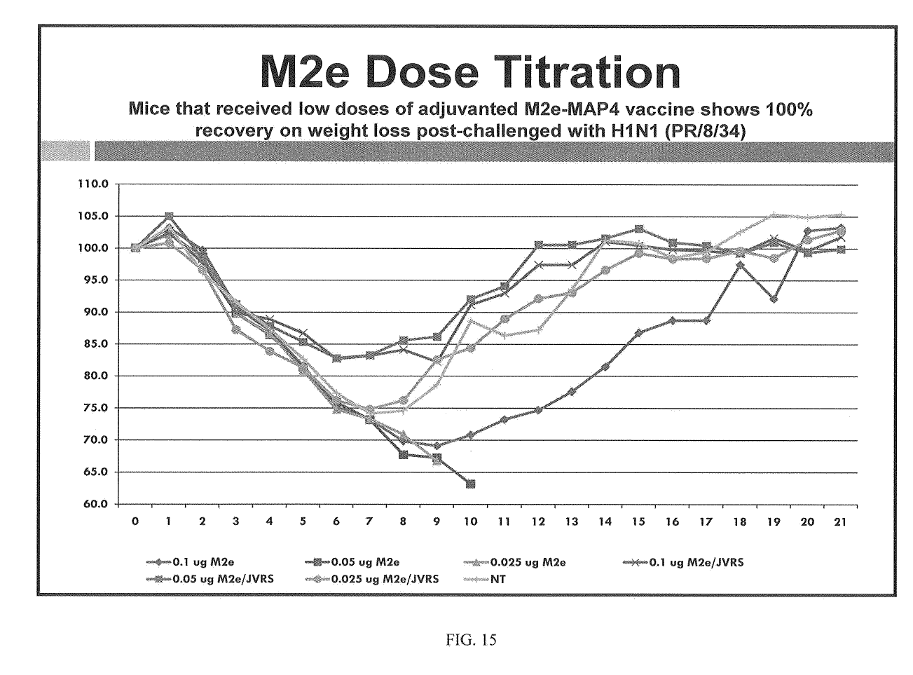

Given the degree of weight loss experienced by mice challenge in experiments such as demonstrated in FIGS. 12 and 13, the pathological effects associated with influenza infection were determined to ensure that M2e-MAP4/JVRS-100 vaccinated and subsequently infected mice had decreased pathological effects associated with influenza infection. Initially, mice were vaccinated on day 0, 14, and 28 with M2e-MAP4/JVRS-100 and challenged with 10×LD₅₀HK×31. At day 4 post-infection, untreated and vaccinated mice were sacrificed and lungs were evaluated for pathological changes consistent with influenza infection. As seen in FIG. 14, lung tissue density is markedly increased in the untreated animal due to accumulation of inflammatory cells within alveolar walls, collapse of alveoli and presence of inflammatory cells mixed with necrotic debris (arrow) within airways.

To examine long-term pathological changes in adjuvanted versus unadjuvanted vaccination, animals were vaccinated with M2e-MAP4 or M2e-MAP4/NRS-100 on day 0, 14, 28 and challenged with 10×LD₅₀ on day 42. Twenty-eight days following lethal challenge, lungs were collected from surviving mice and % of lung involved with lesions were evaluated by a blinded veterinary pathologist. Lungs from mice that received M2e-MAP4/JVRS-100 had significantly fewer and less severe lesions than lungs from mice that received M2e-MAP4 without JVRS-100 (FIG. 6).

Adoptive Transfer of Immune Sera from M2e-MAP4/JVRS-100 Vaccinated Mice (Shown in FIG. 7)

To demonstrate that the mechanism of protection of M2e-MAP4/JVRS-100 vaccine was primarily antibody-mediated; mice were vaccinated with M2e-MAP4/JVRS-100 at day 0, 14, and 28. At day 42, serum was collected from vaccinated and naïve mice and 300 μl adoptively transferred to naïve Balb/c mice. One day following adoptive transfer, mice were challenged with 2×LD₅₀ of H1N1 (PR/8/34) and monitored for survival and body weight loss. Mice adoptively transferred serum from M2e-MAP4/JVRS-100 vaccinated mice had on average less than 10% weight loss and no mortality compared with mice which received adoptively transferred serum from naïve mice which had significant morbidity and 100% mortality following lethal influenza challenge (FIG. 8). In addition, splenocytes were collected from serum donor mice and restimulated with M2e, M2e-MAP4, heat inactivated H1N1 (PR/8/34-40 HA/ml), and live H1N1 (PR/8/34-40 HA/ml) in vitro. None of these conditions resulted in any release of interferon-gamma from splenocytes from vaccinated or naïve mice based on assays with the limit of detection of 7.5 pg/ml (data not shown). These data strongly suggest that the protection afforded by M2e-MAP4/JVRS-100 vaccination is due to an enhanced antibody response.

Dose Titration of M2e-MAP4 with Constant JVRS-100

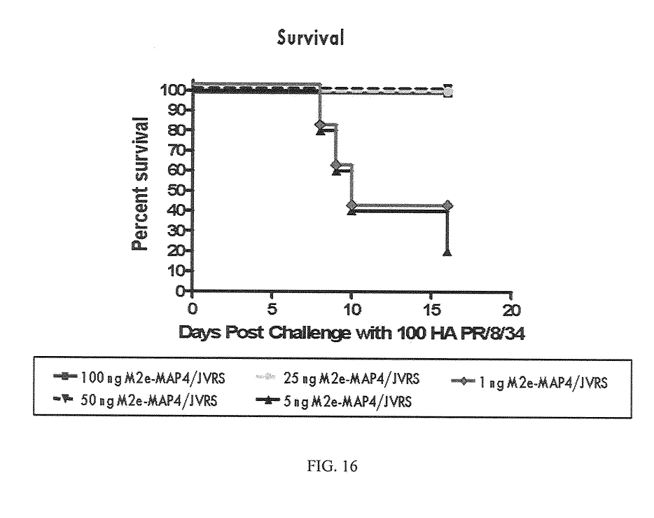

A major impediment of M2e-based vaccines has been a lack of immunogenicity. After determining that doses from 0.1 μg to 5 μg were 100% protective from mortality in lethal challenge (data not shown), a study was conducted in which mice were vaccinated on day 0, 14, and 28 with M2e-MAP4/JVRS-100 with M2e-MAP4 at 100, 50, 25, 5, and 1 ng per dose and challenged with 2×LD₅₀H1N1 (PR/8/34) on day 42. Doses of 100, 50, and 25 ng resulted in 100% survival compared with 5 ng and 1 ng which resulted in 20% and 40% survival, respectively (FIG. 16). Groups of no treatment and unadjuvanted controls in these experiments resulted in 0%-10% survival (data not shown). It is important to note that the 25 ng dose is greater than two orders of magnitude lower than previous investigators have used in vaccination and challenge studies.

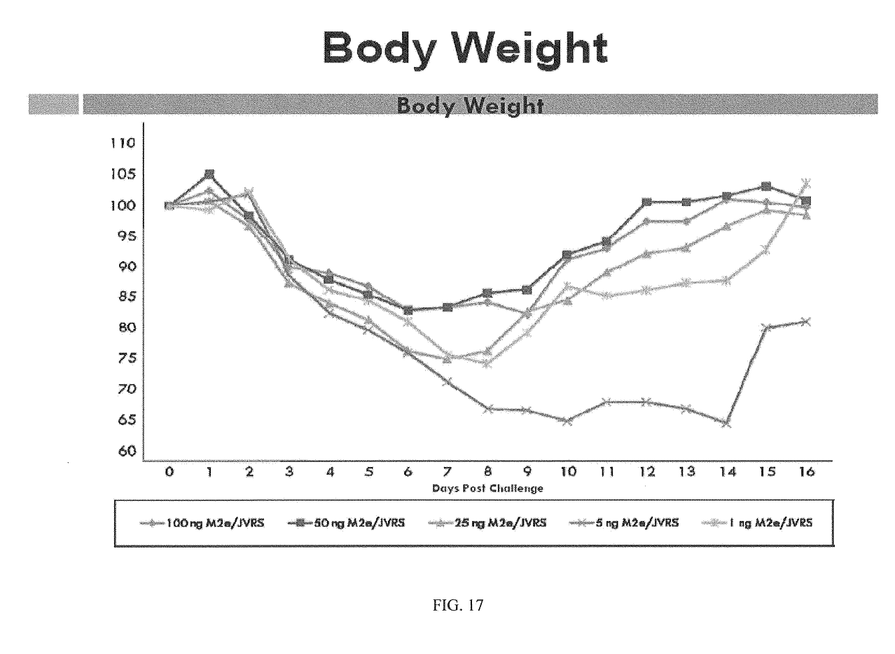

While there was not a difference in survival in the 100, 50, and 25 ng M2e groups, the 25 ng group did show an increase in weight loss compared with the 100 and 50 ng dose groups (FIG. 17). While the 25 ng group did eventually recover to the same level of body weight as the higher dose groups by day 16, there was an approximately 10% difference in the weight loss at day 7 post-infection indicating more advanced disease in this dose group.

Competitive Binding ELISA

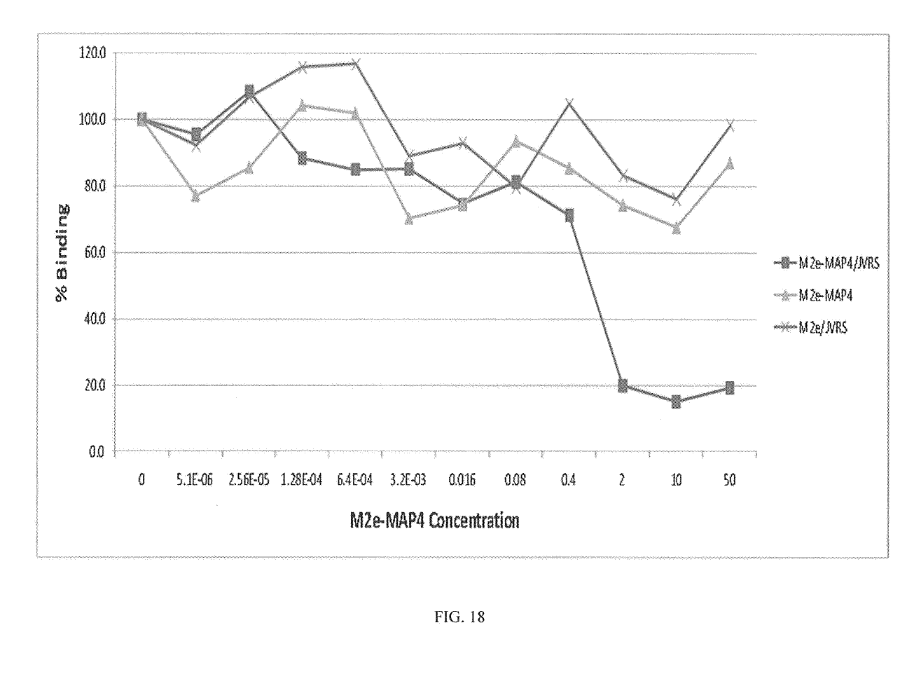

To estimate the potential for conformational antibodies (antibodies versus more than 1 copy of M2e) a competitive binding ELISA was used. Sera from mice vaccinated with M2e in the monomer or tetramer orientation were evaluated in a competitive binding ELISA to determine the extent of binding to fixed influenza infected cells which should be expressing native M2. Briefly, plates were coated with MDCK cells with or without influenza infection and fixed with 80% acetone similar to the final step in an influenza microneutralization antibody titer assay. Sera from mice vaccinated with M2e/JVRS-100, M2e-MAP4, or M2e-MAP4/JVRS-100 were first absorbed on the uninfected plates to remove non-specific antibodies and then mixed with an increasing concentration of M2e-MAP4 before applying to the influenza infected cell coated plates. After incubation plates were washed, mouse anti-IgG antibody HRP conjugate added and ultimately analyzed spectrophotometrically for reduction of substrate by HRP. If conformational epitopes exist they should compete for binding between the free peptide and the plate-bound influenza virus infected cells with a concurrent reduction in the antibody titer detected by ELISA. As can be seen in FIG. 18 below there was a greater reduction of the signal elicited by M2e-MAP4/JVRS-100 versus M2e-MAP4 or M2e/JVRS-100 when competitive binding was assessed with M2e-MAP4. This result suggests that there are antibodies present in mice vaccinated with M2e-MAP4/NRS-100 which are not present in mice vaccinated with the M2e-MAP4 or M2e peptide. Furthermore, the enhanced competitive binding using M2e-MAP4 suggests these antibodies recognize tetrameric forms of M2e which are present in influenza infected cells and both the adjuvant and tetrameric antigen are essential for eliciting these conformational antibodies which results in enhanced recognition of expressed M2e.

Efficacy of M2e-MAP4/TIV/JVRS-100 Vaccine in Mice

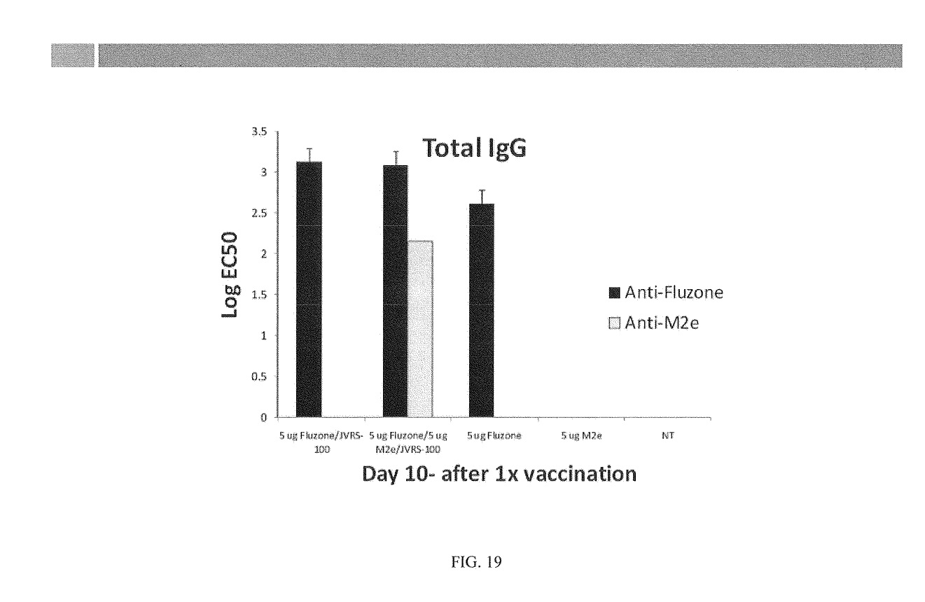

To test the synergistic effect of adding M2e-MAP4 to TIV/NRS-100, we added 5 μg M2e-MAP4 to 5 μg TIV (Fluzone® influenza virus vaccine by Sanofi-Pasteur) and 10 μg JVRS-100 and challenged with a drifted H3N2 virus (HK×31) at 2×LD₅₀ at day 14 following vaccination. As can be seen in FIG. 19, IgG antibody titers were measurable at day 10 after vaccination for TIV/NRS-100; M2e-MAP4/TIV/NRS-100 and TIV alone but not for M2e-MAP4 alone. Furthermore, the addition of 5 μg M2e-MAP4 did not decrease the immune response to 5 μg Fluzone/10 μg JVRS-100.

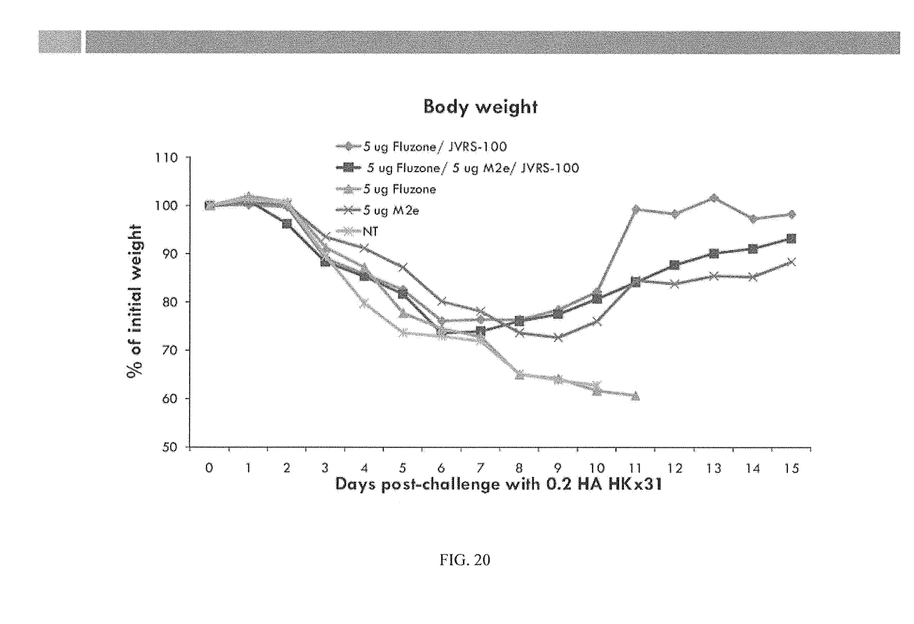

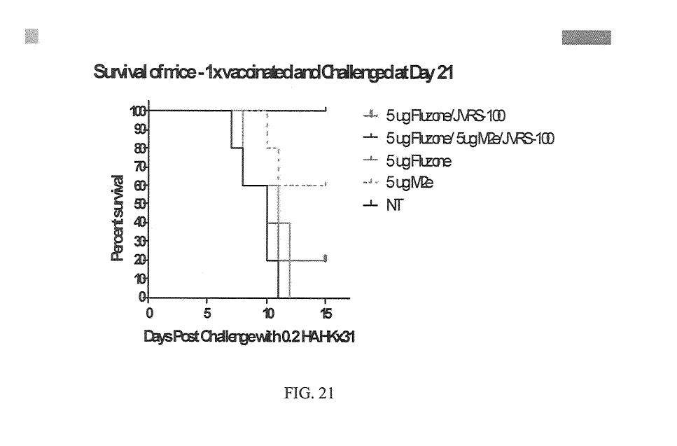

At 21 days following a single vaccination, mice were challenged with 2×LD₅₀ of HK×31 (H3N2) and followed for weight loss (shown in FIG. 20) and survival (shown in FIG. 21). Mice that received a single injection of Fluzone®/M2e-MAP4/JVRS-100 were completely protected from mortality as compared with 60% survival with M2e only, 20% survival with Fluzone®/JVRS-100, and 0% survival for Fluzone® only and no treatment control groups (FIG. 21). These mice, however, were not protected from morbidity associated with influenza infection as represented by body weight loss following challenge (FIG. 20), indicating that the combination of M2e/Fluzone/NRS-100 and drifted challenge required infection to be protective. This is consistent with the hypothesized ADCC mechanism of M2e and the likely T-cell mediated protection afforded by Fluzone®/JVRS-100 vaccination.

Furthermore, we have shown that the inclusion of the M2e-MAP4 with TIV/JVRS-100 increases the survival following challenge with a drifted influenza strain, suggesting that the addition of M2e-MAP4 to adjuvanted TIV may be a successful strategy to prevent morbidity and mortality to mismatched epidemic and pandemic strains of influenza.

M2e-BM2 Fusion Peptide Experiments

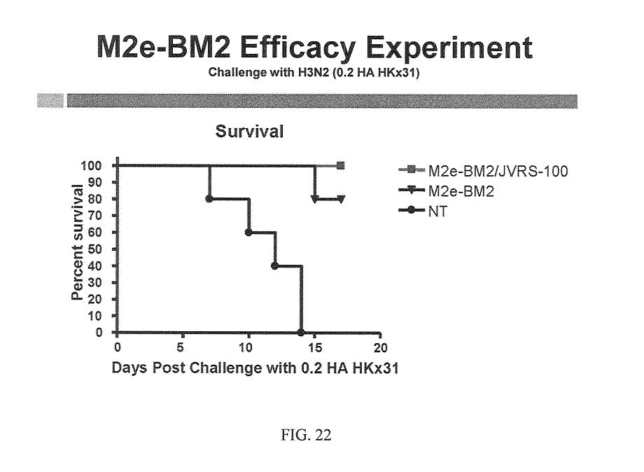

M2e is the conserved peptide portion in Influenza A while BM2 is found in Influenza B. Portions of each were fused together to evaluate the fusion peptides protectiveness on both influenza A and B types. Studies were performed similar to above measuring survival and body weight after vaccination using a M2e-BM2 fusion peptide in MAP-4 configuration and challenge with a lethal influenza antigen. Mice were vaccinated three times at two week intervals IM with M2e-BM2/MAP-4 fusion peptide with and without NRS-100. Two weeks after last vaccination mice were challenged with either PR/8/34, HK×31 or B/Malaysia influenza antigen. The results in general showed improvements of survival and decent mortality profile against challenge with different flu strains. (See FIGS. 22-27.)

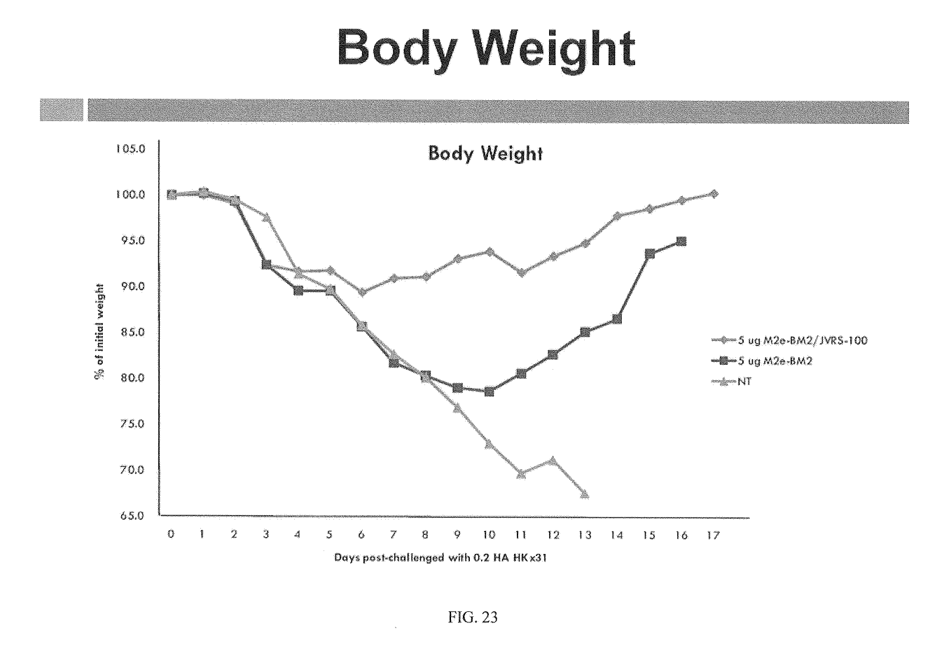

As demonstrated in FIGS. 22 and 23 when challenged with HK×31 M2e-BM2/MAP-4 with and without NRS-100 showed increased survival (See FIG. 22) and improved morbidity (See FIG. 23) with the M2e-BM2/MAP-4 both with and without JVRS-100. Although both parameters had the best results when the M2e-BM2/MAP-4 included JVRS-100.

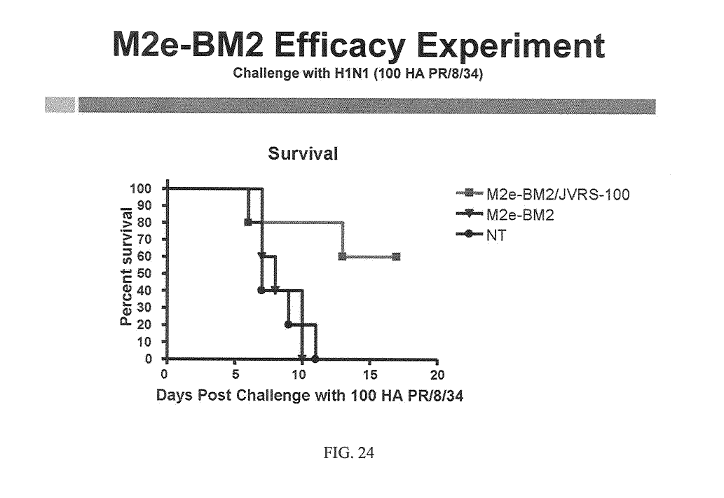

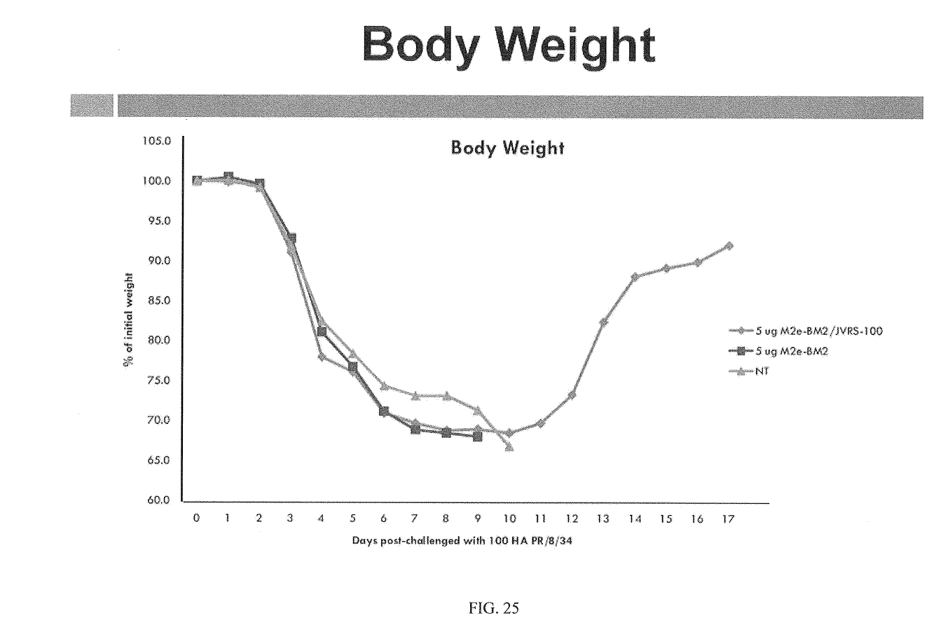

As demonstrated in FIGS. 24 and 25 when challenged with PR/8/34 M2e-BM2/MAP-4 with JVRS-100 showed increased survival (See FIG. 24) but M2e-BM2/MAP-4 without JVRS-100 did not. Additionally BM2/MAP-4 with JVRS-100 showed increased improved morbidity (See FIG. 25) and the M2e-BM2/MAP-4 without JVRS-100 did not.

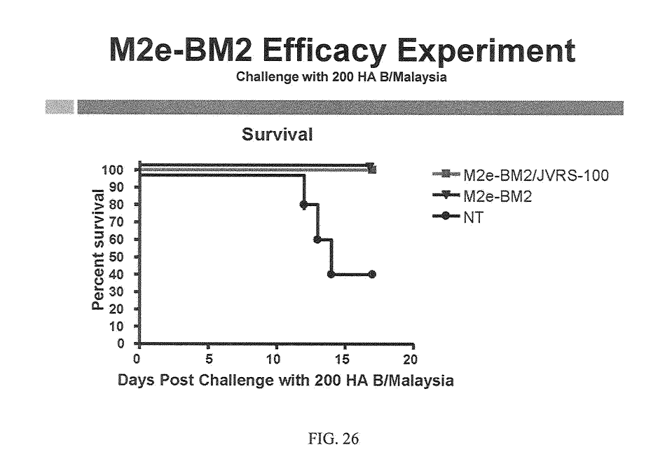

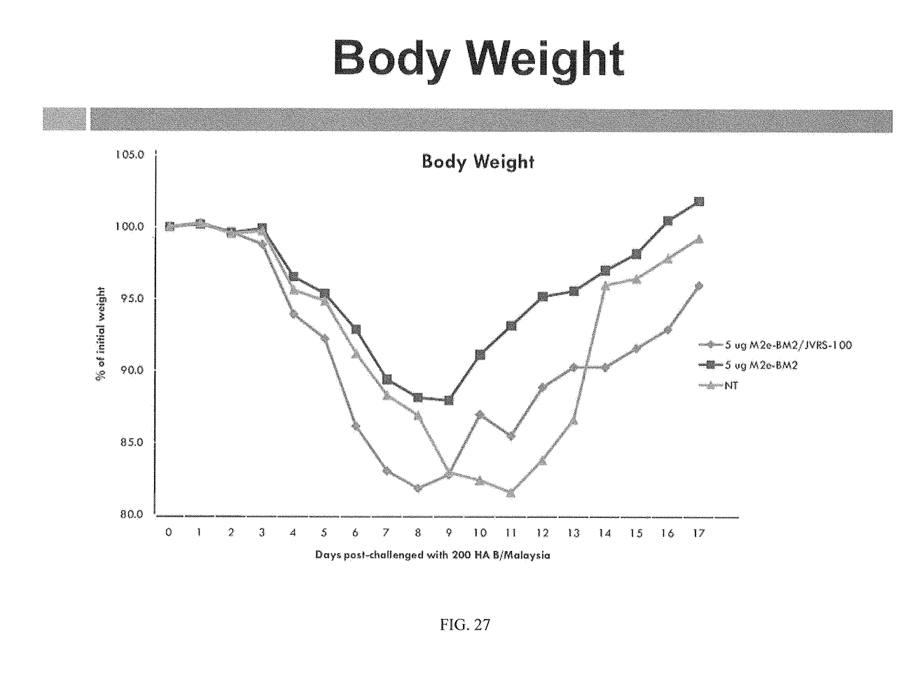

As demonstrated in FIGS. 26 and 27 when challenged with HK×31 M2e-BM2/MAP-4 with and without JVRS-100 showed complete survival (See FIG. 26) and improved morbidity (See FIG. 23) with the M2e-BM2/MAP-4 both with and without JVRS-100. Although morbidity as measured by body weight had the best results when the M2e-BM2/MAP-4 included JVRS-100.

Preparation of Cationic Liposome Delivery Vehicles

The preparation of cationic liposome delivery vehicles such as JVRS-100 is described in U.S. Pat. No. 6,693,086 and below.

The cationic liposomes contemplated consist of DOTAP (1,2 dioleoyl-3-trimethylammonium-propane) and cholesterol mixed in a 1:1 molar ratio, dried down in round bottom tubes, then rehydrated in 5% dextrose solution (D5W) by heating at 50° C. for 6 hours, as described previously (Solodin et al., 1995, Biochemistry 34:13537-13544, incorporated herein by reference in its entirety). Other lipids (e.g., DOTMA) are also contemplated. This procedure results in the formation of liposomes that consists of multilamellar vesicles (MLV), which the present inventors have found give optimal transfection efficiency as compared to small unilamellar vesicles (SUV). The production of MLVs and related “extruded lipids” is also described in Liu et al., 1997, Nature Biotech. 15:167-173; and Templeton et al., 1997, Nature Biotech. 15:647-652; both of which are incorporated herein by reference in their entirety.

Previous Human Clinical Experience with JVRS-100 as an Adjuvant

The initial study of the use of JVRS-100 as an adjuvant was a randomized, double-blind, controlled phase I trial to evaluate the safety, tolerability, and immunogenicity of Fluzone® vaccine mixed with JVRS-100 adjuvant. Eligible volunteers were randomly assigned to one of 12 groups within four ascending cohorts. Within each cohort, volunteers were randomly selected to receive a constant one-half dose of Fluzone® vaccine (22.5 μg) with JVRS-100 adjuvant (7.5 μg, 25 μg, 75 μg, or 225 μg) or Fluzone® vaccine alone at full dose (45 μg) (licensed vaccine manufactured by sanofi pasteur, Swiftwater, Pa., for the 2007-2008 season). One hundred twenty eight (128) adults (male and female) 18-49 years of age, inclusive, were recruited into the study.

The study was designed to determine the dose response of JVRS-100 adjuvant using a sub-optimal (22.5 μg) dose of antigen (Fluzone®). The rationale for the use of a suboptimal dose of antigen is that it potentially increased the sensitivity to detect adjuvant activity, as measured by an increased immunological response. The effect of adjuvants (in general) is also to reduce the amount of antigen needed to achieve a protective immune response. Therefore, the use of half-dose antigen in this trial may demonstrate the dose-sparing effect of JVRS-100. The standard 45 μg dose of Fluzone® is used as a control, allowing a comparison of the immune response to half-dose Fluzone® (with and without adjuvant) to the response to standard influenza vaccination.

Overall JVRS-100 was well tolerated at all dose levels. Adverse events were seen at the higher dose levels (≧75 μg), were predominantly Grade 1 (mild), were of short duration, and were characterized by local injection site symptoms and systemic symptoms suggestive of an acute phase reaction.

The principal efficacy findings were an increase in HAI, neutralizing antibody, and T cell responses associated with JVRS-100 adjuvant. The increase in antibody response was seen principally in the comparison of GMT on Day 28 and GMT fold-increase (Day 0 to 28) for influenza A antigens between adjuvanted and unadjuvanted Fluzone® treatment groups. The increase in GMT and GMT fold-increase was evident at the lowest dose of JVRS-100 (7.5 μg), whereas higher doses did not enhance (or even suppressed) the antibody response. The increase in T cell responses (measured by intracellular cytokine staining, ICS) associated with JVRS-100 was observed for both influenza A and B viruses, and involved both CD4⁺ and CD8⁺ cells secreting interferon-γ, IL-2, TNF-α, and all three cytokines (polyfunctional T cells).

Multiple Antigenic Peptide Sequences

The following table includes examples of peptides and individual sequences contemplated in the present disclosure.

| Peptide | Sequence |
|---|---|
| M2e | SEQ ID NO 1: |
| | SLLTEVETPIRNEWGCRCNDSSD |
| HA0-MAP (15-mer) | SEQ ID NO 2: |
| | (NIPSIQSRGLFGAIA)4-MAPS |
| HAO-MAP (19-mer) | SEQ ID NO 3: |
| | (NIPSIQSRGLFGAIAGFIE)4-MAPS |
| BM2 | SEQ ID NO 4: |
| | (MLEPFQILSICSFILSALHFMAWTIGH)2- |
| | Lys-CONH2 |
| M2e-BM2 fusion | SEQ ID NO 5: |
| peptide | (MLEPFQILPIRNEWGCRCNDSSD) |

Summary of Results

JVRS-100 is an efficient and potent adjuvant that offers advantages in converting a simple, conserved, and minimally immunogenic peptides to highly effective vaccines. The native M2e is a 23-amino acid long ectodomain of the Matrix protein 2 (M2) which is vastly conserved amongst human influenza A virus strains. The synthetic M2e-peptide is constructed in a multiple antigenic peptide (MAP-4) context containing 4-copies of the antigen which presented it in a much more immunogenic form to the immune system. Vaccination with M2e-MAP4/JVRS-100 resulted in a significant increase in total IgG, IgG1 and IgG2a M2e-specific antibodies. As has been shown in previous studies with other antigens, NRS-100 increased the Th1 bias indicated by production of significant amount of anti-M2e IgG2a, which is much more effective at ADCC than IgG1. The addition of JVRS-100 adjuvant protected mice from lethal challenge against H1N1 and H3N2 strains in terms of survival and improved morbidity. The adjuvanted M2e-based vaccine has demonstrated protective immunity primarily due to humoral response and is transferable by serum. The addition of JVRS-100 to M2e-MAP4 showed complete protection at peptide doses of M2e of 100, 50, and 25 ng respectively. This is approximately 40 times less than reported in the literature, indicative of the potency of the JVRS-100/M2e-MAP4 vaccine.

The application of JVRS-100 as an adjuvant to the conserved M2e peptide has made the M2e highly immunogenic, therefore eliciting robust protective response. JVRS-100 is a potent adjuvant when combined with M2e peptide, delivering broad-spectrum protection after challenged with heterotypic Influenza A strains through induction of protective antibodies. The data demonstrates the role of JVRS-100 adjuvant on the development of a Universal Influenza A vaccine in the event of an unmatched seasonal vaccine or an influenza pandemic.

Although the invention has been described with reference to the above example, it will be understood that modifications and variations are encompassed within the spirit and scope of the invention. Accordingly, the invention is limited only by the following claims.

All references and articles cited herein are incorporated by reference.

What is claimed is:

1. A composition useful for vaccinating a mammalian subject against influenza virus comprising one or more multiple antigenic influenza virus peptides formulated with a cationic liposome delivery vehicle.

2. A composition useful for vaccinating a mammalian subject against influenza A comprising multiple antigenic peptide M2e conjugated with an cationic liposome delivery vehicle.

3. The composition of claim 2 wherein the M2e peptide sequence comprises SEQ ID NO: 1.

4. The composition of claim 2 further comprising multiple antigenic peptide HA0.

5. The composition of claim 2 further comprising multiple antigenic peptide BM2.

6. The composition of claim 2 further comprising antigenic peptides HA0 and BM2.

7. A composition useful for vaccinating a mammalian subject against influenza A comprising a fusion peptide conjugated with a cationic liposome delivery vehicle wherein said fusion peptide comprises an amino acid portion of the M2e peptide and an amino acid portion of the BM2 peptide.

8. The composition of claim 7 wherein said fusion peptide comprises 10 to 22 amino acids native to the M2e antigenic peptide and 2 to 12 amino acids native to the BM2 antigenic peptide.

9. The composition of claim 8 wherein said fusion peptide comprises 16 amino acids native to the M2e antigenic peptide and 7 amino acids native to the BM2 antigenic peptide.

10. The composition of claim 7 wherein the fusion peptide sequence comprises SEQ ID NO: 5.

11. The composition of claim 7 further comprising antigenic peptides HA0, BM2 and M2e.

12. A composition useful for vaccinating a mammalian subject against influenza B comprising antigenic peptide BM2 conjugated with a cationic liposome delivery vehicle.

13. The composition of claim 12 wherein the BM2 peptide sequence comprises SEQ ID NO: 4.

14. A composition useful for vaccinating a mammalian subject against influenza B comprising a fusion peptide conjugated with an cationic liposome delivery vehicle wherein said fusion peptide comprises an amino acid portion of the M2e peptide and an amino acid portion of the BM2 peptide.

15. The composition of claim 14 wherein said fusion peptide comprises 10 to 22 amino acids native to the M2e antigenic peptide and 2 to 12 amino acids native to the BM2 antigenic peptide.

16. The composition of claim 15 wherein said fusion peptide comprises 16 amino acids native to the M2e antigenic peptide and 7 amino acids native to the BM2 antigenic peptide.

17. The composition of claim 7 wherein the fusion peptide sequence comprises SEQ ID NO: 5.

18. A method for vaccinating a mammalian subject against influenza virus comprising administering to the subject one or more multiple antigenic influenza virus peptide sequences formulated with a cationic liposome delivery vehicle.

19. A method for vaccinating a subject against influenza A or influenza B virus comprising administering to the subject a composition comprising one or more peptides selected from M2e, HA0 and BM2, or a M2e-BM2 fusion peptide formulated with a cationic liposome delivery vehicle.

20. The method of claim 19 wherein said subject is vaccinated against influenza A and said peptide is M2e.

21. The method of claim 19 wherein said subject is vaccinated against influenza A and said peptides are M2e and HA0.

22. The method of claim 19 wherein said subject is vaccinated against influenza A and said peptides are M2e, HA0 and BM2.

23. The method of claim 19 wherein said subject is vaccinated against influenza A and said peptide is HA0.

24. The method of claim 19 wherein said subject is vaccinated against influenza A and said peptides are HA0 and BM2.

25. The method of claim 19 wherein said subject is vaccinated against influenza B and said peptide is BM2.

26. The method of claim 19 wherein said subject is vaccinated against influenza B and said peptides are BM2 and HA0.

27. The method of claim 19 wherein said subject is vaccinated against influenza B and said peptides are M2e and BM2.

28. The method of claim 19 wherein said subject is vaccinated against influenza B and said peptides are M2e, HA0 and BM2.

29. A vaccine composition comprising:
a. cationic liposome delivery vehicle; and
b. one or more peptides selected from the group consisting of:
i. M2e;
ii. HA0;
iii. BM2; and
iv. a M2e-BM2 fusion peptide.

30. The composition of claim 29 wherein said liposome delivery vehicle comprises lipids selected from the group consisting of multilamellar vesicle lipids and extruded lipids.

31. The composition of claim 29 wherein said liposome delivery vehicle comprises multilamellar vesicle lipids.

32. The composition of claim 29 wherein said liposome delivery vehicle comprises pairs of lipids selected from the group consisting of DOTMA and cholesterol; DOTAP and cholesterol; DOTIM and cholesterol; and DDAB and cholesterol.

33. The composition of claim 32 wherein said liposome delivery vehicle comprises DOTAP and cholesterol.

34. A method for vaccinating a mammal against influenza comprising administering to said mammal an amount of composition effective to prevent or reduce the effects of the influenza virus, wherein said composition comprises:
a. cationic liposome delivery vehicle; and
b. one or more peptides selected from the group consisting of:
i. M2e;
ii. HA0;
iii. BM2; and
iv. a M2e-BM2 fusion peptide.

35. The method of claim 34 wherein said liposome delivery vehicle comprises lipids selected from the group consisting of multilamellar vesicle lipids and extruded lipids.

36. The method of claim 34 wherein said liposome delivery vehicle comprises multilamellar vesicle lipids.

37. The composition of claim 34 wherein said liposome delivery vehicle comprises pairs of lipids selected from the group consisting of DOTMA and cholesterol; DOTAP and cholesterol; DOTIM and cholesterol; and DDAB and cholesterol.

38. The composition of claim 37 wherein said liposome delivery vehicle comprises DOTAP and cholesterol.

39. A composition comprising one or more multiple antigenic influenza virus peptides formulated with a cationic lipid DNA complex.
